(19) United States
(12) Patent Application Publication
(10) Pub. No.: US 2002/0156065 A1
(43) Pub. Date: Oct. 24, 2002

(54) Medicament and method for the remediation of aberrant fibrotic tissue masses with topical calcium channel blocker preparations

(76) Inventor: W. Jerry Easterling, San Antonio, TX (US)

Correspondence Address: DAVID G. HENRY, 900 Washington Avenue, P.O. Box 1470, Waco, TX, 76701, US

(21) Appl. No.: 10/072,654

(22) Filed: Feb. 8, 2002

Related U.S. Application Data
(63) Continuation-in-part of application No. 09/514,796, filed on Feb. 28, 2000, now abandoned.
Application No. 09/514,796 is a continuation-in-part of application No. 09/128,103, filed on Aug. 3, 1998, now Pat. No. 6,031,005.

(51) Int. Cl. A61K 31/554

(52) U.S. Cl. 514/211.07

(57) ABSTRACT

The invention is of a topical medicament and associated methodology for use thereof, through the use of which sub-dermal, aberrant fibrotic tissue masses are “remodeled” through the topical, non-invasive, transdermal application of the calcium channel blocker diltiazem.

### CITATION TO PRIOR APPLICATION

This is a continuation-in-part with respect to U.S. application Ser. No. 09/514,796 filed Feb. 28, 2000, which was a continuation-in-part of U.S. application Ser. No. 09/128,103 (now U.S. Pat. No. 6,031,005), from which application and its parent application priority is here claimed under 35 U.S.C. §120.

### BACKGROUND OF THE INVENTION

1. Field of the Invention

Applicant's invention relates to medicaments and treatment procedures relating to such conditions as arise from aberrant fibrotic tissue masses or structures, including Peyronie's disease, Dupuytren's Hand Contracture, Ledderhose Fibrosis, scars, and even “cellulite.”

2. Background Information

In U.S. Pat. No. 6,031,005 (and subsequently filed continuation-in-part applications in relation thereto, which CIPs have not issued at the time of this filing), the present inventor has provided new and unobvious treatment regimens for a variety of fibrotic conditions through the use of topically applied calcium channel blocker-based preparations. The specification of U.S. Pat. No. 6,031,005 (“the '005 patent”) is incorporated herein by reference, as if set forth herein verbatim.

The preparations and associated methods for the topical application of calcium channel blocker preparations as taught in the '005 patent have proven remarkably effective in treating, not only the conditions described in the '005 patent (notably Peyronie's disease, Dupuytren's Hand Contracture, Ledderhose Fibrosis and scarring), but also in treating hemangiomas, “spider veins” and “cellulite.” However, the previously preferred embodiment and best known mode of the topical calcium channel blocker preparations of the '005 patent's invention (the verapamil-based composition as taught in the '005 patent and continuations thereof) have shown to cause skin irritation for certain patients. In addition, certain limited numbers of Peyronies disease patients have, after marked, but incomplete remediation of their conditions, reached plateaus beyond which additional treatment with the verapamil-based formulations proved largely ineffective, or at least marked deceleration.

Although diltiazem was mentioned in the very first patent application filed in this series as one alternative to verapamil, the present inventor had not yet determined (as he has now) that such calcium channel blocker has two actual advantages over verapamil in some instances: (1) a much reduced tendency to cause skin irritation in patients with demonstrated sensitivity to verapamil; and (2) diltiazem has proven efficacious in rare cases where verapamil was or became ineffective. To date, the present inventor has not found that diltiazem is inferior in any way in the treatment of conditions for which verapamil has been used in his studies and trials as reflected in the prior patents and patent applications which preceded this present filing.

### SUMMARY OF THE INVENTION

It is an object of the present invention to provide an improved medicament useful in the treatment of aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”).

It is another object of the present invention to provide an improved medicament useful in the treatment of aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which medicament has as its primary active ingredient, a compound which is, at least with certain patients and certain conditions, a more efficacious compound than previously utilized compounds;

It is another object of the present invention to provide an improved medicament useful in the treatment of aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which medicament has as its primary active ingredient, a compound which is, at least with certain patients and certain conditions, a more efficacious compound than verapamil.

It is an object of the present invention to provide an improved medicament useful in the treatment of aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which medicament is applications when topically applied and thereby obviates the need for invasive treatment regimens for such conditions.

It is another object of the present invention to provide and improved method for treating aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”).

It is another object of the present invention to provide and improved method and medicament for treating severe burns whereby the injury is managed such that dermal rupturing and subsequent scarring are mitigated.

It is another object of the present invention to provide and improved method for treating aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which method involves the use of an improved medicament having as its primary active ingredient, a compound which is, at least with certain patients and certain conditions, a more efficacious compound than previously utilized compounds;

It is another object of the present invention to provide and improved method for preparing a topical calcium channel blocker preparation for use in treating aberrant fibrotic tissue manifestations which are exemplified by sub-dermal plaque or scar tissue formation or accumulations, such as Peyronie's disease, Dupuytren's contracture, Ledderhose Fibrosis, scars, hemangiomas, and lipoederma (“cellulite”), which method involves the use of a medicament which has as its primary active ingredient, a compound which is, at least with certain patients and certain conditions, a more efficacious compound than verapamil.

In satisfaction of these and related objectives, Applicant's present invention provides an improved topical medicament and associated methodologies for preparation and use thereof, through the use of which medicament, through topical application, aberrant fibrotic tissue manifestations exemplified by sub-dermal plaque or scar tissue formation or accumulations may be treated, such conditions including, without limitation, Peyronie's disease, Dupuytren's contracture, Ledderhose Fibrosis, existing scars, hemangiomas, and lipoederma (“cellulite”). The present medicament also shows great promise in treating severe bums. The medicament uses, as its primary active ingredient, the calcium channel blocker diltiazem, in most cases, as a replacement for verapamil, upon which the present inventor's prior inventions were preferentially based.

### DETAILED DESCRIPTION OF THE PREFERRED EMBODIMENT

In the preferred embodiment of the present medicament, and in the medicament upon which the associated method are based, the primary active ingredient the benzothiazepine diltiazem. However, it should be understood that other calcium channel blockers of inconsequentially differing structure or nature may be substituted, and yet still fall within the scope of the present invention and the claims covering same.

I. Preparation.

The now-preferred diltiazem-based gels of the present invention may be prepared according to the following disclosure and protocol, with variations appropriate to a desired scale of production as will be apparent to persons skilled in the production of pharmaceutical preparations:

A. Constituents of Preferred Embodiment of Topical Diltiazem Gel

| Ingredients | (% W/W) |
|---|---|
| Diltiazem | 16.00 |
| Poloxamer | 13.70 |
| Soya Lecithin | 13.25 |
| Isopropyl Myristate | 13.25 |
| Potassium Sorbate | 0.14 |
| Sorbic Acid | 0.08 |
| Edetate Disodium | 0.01 |
| Butylated Hydroxy Toluene | 0.10 |
| Purified Water | 43.47 |

B. Preparation of Pluronic Gel 20% (To Make 3000 Gm)

| Ingredients | Quantity |
|---|---|
| Pluronic F127 NF (Poloxamer 407) | 600.00 Gm |
| Potassium Sorbate NF | 9.00 Gm |
| Water (Sterile for Irrigation) qs to | 3,000.00 Gm |

Directions: Prepare a pluronic gel by combining the potassium sorbate and pluronic F 127 and bringing to a total weight of 3,000 Gm. with cold (refrigerated) sterile water.

Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight.

The above solution will solidify into a clear gel at room temperature.

C. Preparation of Pluronic Gel 30% (To Make 2000 Gm).

| Ingredients | Quantity |
|---|---|
| Pluronic F 127 NF (Poloxamer 407) | 600.00 Gm |
| Potassium Sorbate NF | 6.00 Gm |
| Water (Sterile for Irrigation) qs to | 2,000.00 Gm |

Instructions: Prepare a pluronic gel by combining the potassium sorbate and pluronic F 127 and bringing to a total weight of 2,000 Gm. with cold (refrigerated) sterile water. Make sure that all the granules are wet, and place in a refrigerator. Mixture will form a clear solution over 24-48 hours.

Alternate Procedure: The above mixture can be uniformly mixed with a mixing blade. It will take on the appearance of beaten egg whites. When placed in the refrigerator it will form a clear solution much faster, usually overnight. The above solution will solidify into a clear gel at room temperature.

D. Preparation of Lecithin/Isopropyl Myristate Solution
(To Make 3000 Gm).

| Ingredients | Ouantity |
|---|---|
| Lecithin Soya Granular | 1,494.0 Gm |
| Isopropyl Myristate NF | 1,494.0 Gm |
| Sorbic Acid NF Powder | 9.90 Gm |

Instructions: Disperse lecithin and sorbic acid in isopropyl myristate. Allow to stand at room temperature until a liquid of syrup consistency forms. Stir well and store in a light protected container.

E. Preparation of 160 mg/ml Diltiazem Gel

Add diltiazem to water and stir with the aid of heat (90-100 degrees Centigrade) until diltiazem is completely dissolved and a clear solution exists. Immediately add lecithin/isopropyl myristate and stir well. Add diltiazem phase to pluronic gel and stir with a 3 inch mixing blade at 3100 rpm for 10 minutes. Store in glaminate tubes using nitrogen gas to displace room air from tubes during the filling and sealing processes.

II. Use of Preparations.

Use of the topical calcium channel blocker preparation of the present invention involves simply applying a thin coating of the gel to an affected area or bodily structure, usually once daily (although some clinicians have reported twice daily regimens to be noticeably beneficial with certain “problem cases.”. Clinicians will prescribe certain volumetric dosages, which dosages can be metered by any number of conventional metering means (syringes, dosimeters, blister packs, single-dose tubes, etc.)

In the case of Peyronie's disease, the patient should apply the medication by starting at the point where the plaque is heaviest or where the curvature begins and work out until the entire penile shaft has been covered with medication. In the case of Dupuytren's Contracture, the patient should coat the cords and immediately surrounding areas with the prescribed dosage of the gels, with the same basic approach being applicable to treating Ledderhose fibrosis of the feet. In the case of scars and hemangiomas, the gels are applied to the visible deformation and the immediately surrounding areas. In the case of “cellulite”, the gels are applied generally to the affected area, with caution being exercised with respect to application to large areas of the skin, in order to avoid excessive systemic absorption.

Although the invention has been described with reference to specific embodiments, particularly with respect to the particular active ingredient of the present medicament, this description is not meant to be construed in a limited sense, in particular to limit the scope of the appended claims to cover only those medicaments and associated modalities of treatment which include diltiazem as the calcium channel blocker, the function of which in the area of the remediation of aberrant fibrotic tissue masses appears to lie at the heart of the efficacy of the present medicament. Various modifications of the disclosed embodiments, as well as alternative embodiments of the inventions will become apparent to persons skilled in the art upon the reference to the description of the invention. It is, therefore, contemplated that the appended claims will cover such modifications that fall within the scope of the invention.

I claim:

1. A medicament for use in the remediation of aberrant fibrotic tissue masses comprising:
carrier host agent for facilitating transdermal, non-invasive delivery of a calcium channel blocker agent to an aberrant fibrotic tissue mass;
a benzothiazepine calcium channel blocker agent dissolved in said carrier host agent.

2. The medicament of claim 1 wherein said benzothiazepine calcium channel blocker agent is diltiazem.

3. A method for the remediation of aberrant fibrotic tissue masses comprising the steps of:
selecting a composition comprising:
carrier host agent for facilitating transdermal, non-invasive delivery of a calcium channel blocker agent to an aberrant fibrotic tissue mass;
a benzothiazepine calcium channel blocker agent dissolved or suspended in said carrier host agent;
topically applying said composition to the skin of a recipient patient, which skin overlies an aberrant fibrotic tissue mass.

4. The method of claim 3 wherein said calcium channel blocker means is diltiazem.

5. A medicament for use in the remediation of aberrant fibrotic tissue masses comprising:
carrier host agent for facilitating transdermal, non-invasive delivery of a calcium channel blocker agent to an aberrant fibrotic tissue mass associated with a disease state selected from a group consisting of Peyronie's disease, Dupuytren's Hand Contracture, Ledderhose Fibrosis, lipoederma, and traumatic scarring;
a benzothiazepine calcium channel blocker agent dissolved in said carrier host agent.

6. The medicament of claim 1 wherein said benzothiazepine calcium channel blocker agent is diltiazem.

7. A method for the remediation of aberrant fibrotic tissue masses comprising the steps of:
selecting a composition comprising:
carrier host agent for facilitating transdermal, non-invasive delivery of a calcium channel blocker agent to an aberrant fibrotic tissue mass associated with a disease state selected from a group consisting of Peyronie's disease, Dupuytren's Hand Contracture, Ledderhose Fibrosis, lipoederma, and traumatic scarring;
a benzothiazepine calcium channel blocker agent dissolved or suspended in said carrier host agent;
topically applying said composition to the skin of a recipient patient, which skin overlies an aberrant fibrotic tissue mass.

8. The method of claim 3 wherein said calcium channel blocker means is diltiazem.
